SEQUENCE LISTING (7 sequences)

SEQ ID NO: 1 - protein, 274 aa, Bacillus Subtilis
AQSVPYGISQIKAPALHSQGYTGSNVKVAVIDSGIDSSHPDLNVRGGASFVPSETNPYQDGSSHGTHVAGTIAALNNSIGVLGVAPSASYAVKVLDSTGSGQYSWIINGIEWAISNNMGVINMSLGGPSGSTALKTVVDKAVSSGIVVAAAAGNEGSSGSSSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 2 - protein, 381 aa, Bacillus Subtilis
MRSKKLWISLLFALTLIFTMAFSNMSAQAAGKSSTEKKYIVGFKQTMSAMSSAKKKDVISEKGGKVQKQFKYVNAAAATLDEKAVKELKKDPSVAYVEEDHIAHEYAQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSVGSELDVMAPGVSIQSTLPGGTYGAYNGTSMATPHVAGAAALILSKHPTWTNAQVRDRLESTATYLGSSFYYGKGLINVQAAAQ

SEQ ID NO: 3 - protein, 381 aa, Bacillus Subtilis
MRSKKLWISLLFALTLIFTMAFSNMSAQAAGKSSTEKKYIVGFKQTMSAMSSAKKKDVISEKGGKVQKQFKYVNAAAATLDEKAVKELKKDPSVAYVEEDHIAHEYAQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSVGSELDVMAPGVSIQSTLPGGTYGAYNGTSMATPHVAGAAALILSKHPTWTNAQVRDRLESTATYLGNSFYYGKGLINVQAAAQ

SEQ ID NO: 4 - protein, 381 aa, Bacillus Subtilis; identical to SEQ ID NO: 3

SEQ ID NO: 5 - DNA, 1327 bp, Bacillus Subtilis
gtatgaaaatagttatttcgagctctctacggaaatagcgagagatgatatacctaaatagagataaaatcatctcaaaaaaatgggtctactaaaatattattccatctattacaataaatgcacagaatagtcttttaagtaagtctactctgaattttttaaaaggagagggtaaagagtgagaagcaaaaaattgtggatcagcttgttgtttgcgttaacgttaatctttacgatggcgttcagcaacatgtctgcgcaggctgccggaaaaagcagtacagaaaagaaatacattgtcggatttaagcagacaatgagtgccatgagttccgccaagaaaaaggatgttatttctgaaaaaggcggaaaggttcaaaagcaatttaagtatgttaacgcggccgcagcaacattggatgaaaaagctgtaaaagaattgaaaaaagatccgagcgttgcatatgtggaagaagatcatattgcacatgaatatgcgcaatctgttccttatggcatttctcaaattaaagcgccggctcttcactctcaaggctacacaggctctaacgtaaaagtagctgttatcgacagcggaattgactcttctcatcctgacttaaacgtcagaggcggagcaagcttcgttccttctgaaacaaacccataccaggacggcagttctcacggtacgcatgtcgccggtacgattgccgctcttaataactcaatcggtgttctgggcgtagcgccaagcgcatcattatatgcagtaaaagtgcttgattcaacaggaagcggccaatatagctggattattaacggcattgagtgggccatttccaacaatatggatgttatcaacatgagccttggcggacctactggttctacagcgctgaaaacagtagttgataaagcggtttccagcggtatcgtcgttgctgccgcagccggaaacgaaggttcatccggaagcacaagcacagtcggctaccctgcaaaatatccttctactattgcagtaggtgcggtaaacagcagcaaccaaagagcttcattctccagcgtaggttctgagcttgatgtaatggctcctggcgtgtccatccaaagcacacttcctggaggcacttacggcgcttataacggaacgtccatggcgactcctcacgttgccggagcagcagcgctaattctttctaagcacccgacttggacaaacgcgcaagtccgtgatcgtttagaaagcactgcaacataccttggaagctctttctactatggaaaagggttaatcaacgtacaagcagctgcacaataa

SEQ ID NO: 6 - DNA, 825 bp, Bacillus Subtilis
gcgcaatctgttccttatggcatttctcaaattaaagcgccggctcttcactctcaaggctacacaggctctaacgtaaaagtagctgttatcgacagcggaattgactcttctcatcctgacttaaacgtcagaggcggagcaagcttcgtaccttctgaaacaaacccataccaggacggcagttctcacggtacgcatgtagccggtacgattgccgctcttaataactcaatcggtgttctgggcgtagcgccaagcgcatcattatatgcagtaaaagtgcttgattcaacaggaagcggccaatatagctggattattaacggcattgaatgggccatttccaacaatatgggtgttattaacatgagcctcggcggaccttctggttctacagcgctgaaaacagtcgttgataaagccgtttccagcggtatcgtcgttgctgccgctgcaggaaacgaaggttcgtccggaagctcaagcacagtcggctaccctgcaaaatatccttctactattgcggtaggtgcggtaaacagcagcaaccaaagagcttcattctcaagcgcaggttctgagcttgatgtgatggctcctggcgtatccatccaaagcacacttcctggaggcacttacggtgcttacaacggcacgtccatggcgactcctcacgttgccggagcagcagcgctaattctttctaagcatccgacttggactaacgcacaagtccgtgatcgtttagaaagcactgcaacatatcttggaaactctttctactatggaaaagggttaatcaacgtacaagcagctgcacaa

SEQ ID NO: 7 - DNA, 1146 bp, Bacillus Subtilis
gtgagaagcaaaaaattgtggatcagcttgttgtttgcgttaacgttaatctttacgatggcgttcagcaacatgtctgcgcaggctgccggaaaaagcagtacagaaaagaaatacattgtcggatttaagcagacaatgagtgccatgagttccgccaagaaaaaggatgttatttctgaaaaaggcggaaaggttcaaaagcaatttaagtatgttaacgcggccgcagcaacattggatgaaaaagctgtaaaagaattgaaaaaagatccgagcgttgcatatgtggaagaagatcatattgcacatgaatatgcgcaatctgttccttatggcatttctcaaattaaagcgccggctcttcactctcaaggctacacaggctctaacgtaaaagtagctgttatcgacagcggaattgactcttctcatcctgacttaaacgtcagaggcggagcaagcttcgttccttctgaaacaaacccataccaggacggcagttctcacggtacgcatgtcgccggtacgattgccgctcttaataactcaatcggtgttctgggcgtagcgccaagcgcatcattatatgcagtaaaagtgcttgattcaacaggaagcggccaatatagctggattattaacggcattgagtgggccatttccaacaatatggatgttatcaacatgagccttggcggacctactggttctacagcgctgaaaacagtagttgataaagcggtttccagcggtatcgtcgttgctgccgcagccggaaacgaaggttcatccggaagcacaagcacagtcggctaccctgcaaaatatccttctactattgcagtaggtgcggtaaacagcagcaaccaaagagcttcattctccagcgtaggttctgagcttgatgtaatggctcctggcgtgtccatccaaagcacacttcctggaggcacttacggcgcttataacggaacgtccatggcgactcctcacgttgccggagcagcagcgctaattctttctaagcacccgacttggacaaacgcgcaagtccgtgatcgtttagaaagcactgcaacatatcttggaaactctttctactatggaaaagggttaatcaacgtacaagcagctgcacaataa

(19) United States
(12) Patent Application Publication
(10) Pub. No.: US 2004/0223962 A1
(43) Pub. Date: Nov. 11, 2004

(54) Method and composition for preventing or reducing edema, deep vein thrombosis and/or pulmonary embolism

(76) Inventor: Neil H. Riordan, Tempe, AZ (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/647,131

(22) Filed: Aug. 22, 2003

Related U.S. Application Data
(60) Provisional application No. 60/468,948, filed on May 7, 2003.

(51) Int. Cl. A61K 38/48; A61K 31/385; A61K 35/78; A61K 31/355

(52) U.S. Cl. 424/94.63; 514/456; 514/458; 514/474; 514/440; 424/770

(57) ABSTRACT

The present invention relates to a composition and method for preventing or reducing edema, deep vein thrombosis (DVT), and/or pulmonary embolism by administering a combination of a fibrinolytic agent and an antioxidant. The composition is particularly useful for treating individuals prior to or during long term flights or other situations involving extended immobility.

### RELATED APPLICATION

This application claims priority to U.S. provisional application Ser. No. 60/468,948, which was filed on May 7, 2003, the disclosure of which is hereby incorporated by reference in its entirety.

### BACKGROUND OF THE INVENTION

1. Field of the Invention

The present invention relates to a composition and method for preventing or reducing edema, deep vein thrombosis (DVT), and/or pulmonary embolism by administering a combination of a fibrinolytic agent and an antioxidant. The composition is particularly useful for treating individuals prior to or during long term flights or other situations involving extended immobility.

2. Description of the Related Art

Edema, or swelling of the lower extremities, is commonly observed in almost all individuals, including healthy individuals, when immobilized for long periods. Edema and swelling during aircraft flights are typically caused by immobility of the individual in a cramped position, caused by the limited sitting space available on aircraft flights. This cramped environment, in association with the decreased air pressure in the cabin environment, leads to vein dilation and swelling of the tissues. The problem may be aggravated by the presence of venous disease, diabetic microangiopathy and other conditions causing edema, such as, for example, cardiac and renal insufficiency and anti-hypertensive treatment. In some cases edema may cause the compression of minor veins, which compression may be an important initial cause of DVT (Belcaro G., Circulation, Suppl II, 104:II-528 (2001); Belcaro et al., J Am Coll Cardiol, 39, Suppl 5:212A (2002); Anonymous, American Heart Association Scientific Sessions, 3:8 (2001), the disclosures of which are hereby incorporated by reference in their entireties).

DVT and pulmonary embolism (PE) constitute major health problems in the United States; up to 600,000 hospitalizations a year can be attributed to DVT and PE. Situations that result in extended immobilization of an individual, particularly in a cramped and/or sitting position, have been associated with both DVT and PE. The prevalence of DVT is higher in high-risk subjects, such as, for example, individuals with a past history of DVT, hormonal treatment, malignancy, or recent surgery. Venous thromboembolism is also a significant risk in surgical patient populations where preoperative, operative and postoperative immobilization with concomitant loss of venous pump function causes blood stasis.

Air travelers have a significant risk of developing edema, DVT, or PE. The problem is exacerbated during long airline flights, due to prolonged bending and compression of veins (i.e. popliteal, soleal veins) on the edges of the seat, which may contribute to stasis and thrombosis (Geroulakos et al., Eur J Vasc Endovasc Surg, 20:102-104 (2000); Perry K., Guardian (2001); Homans J., N Engl J Med, 250:148-149 (1954); Symington et al., Br J Chest, 17:138-140 (1977); Simpson K., Lancet, 11:744 (1940); Ferrari et al., Chest, 115:440-444 (1999); Cruickshank et al., Lancet, 2:497-498 (1988); Kraaijenhagen et al, Lancet, 356:1492-93 (2000), the disclosures of which are hereby incorporated by reference in their entireties). It is estimated that 4 to 6% of passengers on a long-haul flight of greater than 10 hours will experience a deep vein thrombosis. Blood concentration, decreased fluid intake, and a dry atmosphere in cabins are among the factors that have been implicated (Cruickshank et al., Lancet, 2:497-498 (1988); Kraaijenhagen et al, Lancet, 356:1492-93 (2000); Sarvesvaran R., Med Sci Law, 26:35-38 (1986), the disclosures of which are hereby incorporated by reference in their entireties). Fibrinogen and fibrinolysis alterations in blood have been reported during both simulated and actual long flights. (Landgraf et al., Aviat Space Envir Med, 65:930-935 (1994); AMA Commission on Emergency Services, “Medical aspects of transportation aboard commercial aircraft,” JAMA 247:1007-1011 (1982), the disclosure of which is hereby incorporated by reference in its entirety). Immobility, lower air pressure, and relative hypoxia alter fibrinolytic activity, causing release of vein wall factors leading to stasis and thrombosis. (Gertler et al., J Vasc Surg, 18:939-946 (1993); Bendz et al., Lancet, 356:1657-1658 (2000), the disclosures of which are hereby incorporated by reference in their entireties). Further evidence linking the incidence of DVT during or after long flights has been reported (Belcaro et al., Angiology, 53:17-21 (2001); Belcaro et al., Angiology, 52:369-74 (2001); Cesarone et al., Angiology, 53:1-6 (2002); Belcaro G., Circulation, Suppl II, 104:II-528 (2001); Belcaro et al., J Am Coll Cardiol, 39, Suppl 5:212A (2002); the disclosures of which are hereby incorporated by reference in their entireties).

What is needed in the art is a method for preventing or treating edema, DVT and PE that occurs during long flights or periods of extended immobilization, using compositions that are able to reduce platelet aggregation and/or dissolve fibrin clots once they have formed.

### SUMMARY OF THE INVENTION

In some embodiments of the invention, a pharmaceutical preparation having effective amounts of a fibrinolytic agent and an antioxidant is provided. In some embodiments of the invention, the fibrinolytic agent may be chosen from nattokinase, urokinase, subtilisin, or plasmin. In additional embodiments of the invention, the antioxidant may be pine bark extract, or may be chosen from the following group: vitamin C, vitamin E, a catechin, a carotenoid, a flavonoid, coenzyme Q10 (ubiquinone), an isoflavone, a phenylpropanoid, a polyphenol, a tocopherol, alpha tocopherol, selenium, magnesium, α-lipoic acid, TBHQ, BHA, BHT, a tocotrienol, ascorbic acid, resveratrol, an oleoresin, rosemary extract, tea extract, grape seed extract, and an antioxidant extract from fruit skin or from a seed.

Other embodiments of the invention provide a pharmaceutical preparation having effective amounts of pine bark extract and nattokinase. The pine bark extract may prepared, for example, from French Maritime Pine, Pinus maritima.

In other embodiments of the invention, methods for decreasing swelling of the lower extremities, edema, pulmonary embolism, or thrombosis by administering a pharmaceutical preparation comprising a fibrinolytic agent and an antioxidant. In preferred embodiments of the invention, the swelling of the lower extremities, edema, pulmonary embolism, or thrombosis is caused by periods of confinement or enforced inactivity. The fibrinolytic agent can be an enzyme, and can further be chosen from urokinase, subtilisin, plasmin, or nattokinase. The antioxidant can be pine bark extract. In some embodiments, the pine bark extract can be from French maritime pine (Pinus maritima), and may be an aqueous extract. In some embodiments of the invention, the extract contains at least one of the following components: bioflavonoid, catechin, epicatechin, taxifolin, oligomeric proanthocyanidnin, phenolic fruit acid, ferulic acid, and caffeic acid. In other embodiments of the invention, the antioxidant can be chosen from vitamin C, vitamin E, a catechin, a carotenoid, a flavonoid, coenzyme Q10 (ubiquinone), an isoflavone, a phenylpropanoid, a polyphenol, a tocopherol, alpha tocopherol, selenium, magnesium, α-lipoic acid, TBHQ, BHA, BHT, a tocotrienol, ascorbic acid, resveratrol, an oleoresin, rosemary extract, tea extract, grape seed extract, and an antioxidant extract from fruit skin or from a seed.

In some embodiments of the invention, the confinement or period of enforced inactivity is due to at least one of the following: airline flight, bus travel, car travel, and train travel. In preferred embodiments of the invention, the confinement is due to an airline flight. In most preferred embodiments, the airline flight is one that has a duration of greater than 6 hours.

### DESCRIPTION OF THE FIGURES

FIG. 1 is a bar graph showing the variation in edema score before and after an airline flight in control individuals or individuals treated with PYCNOGENOL®/nattokinase tablets (“FLITE TABS™”).

### DETAILED DESCRIPTION OF THE PREFERRED EMBODIMENT

The present invention relates to compositions and methods for preventing or reducing edema, deep vein thrombosis (DVT), and/or pulmonary embolism resulting from periods of extended confinement or enforced inactivity, by administering a combination of a fibrinolytic agent and an antioxidant. The composition is particularly useful for treating individuals prior to or during long term flights.

In some embodiments, the invention relates to a combination of natural products that have the ability both to inhibit excessive aggregation of platelets and to dissolve fibrin clots once they have formed. This combination has been found to decrease edema in air passengers on long haul flights. Each product used alone did not have the same effect on peripheral lower extremity edema as the combination of products. Given the unexpected finding that the combination of natural products inhibits edema, this combination of an antioxidant and a fibrinolytic agent should also be effective in preventing deep vein thrombosis, a condition that is closely associated with lower extremity edema.

The results of a study to test the ability of the invention to inhibit the formation of blood clots in individuals on long-haul air flights are disclosed herein in Examples 2 through 13. The aim of this study was to evaluate the preventive effects of one combination formulation of an antioxidant and fibrinolytic agent, “FLITE TABS™,” in long-haul flights (7-8 hours) in subjects at high risk for DVT. FLITE TABS™ (Aidan, Ariz., USA) contain PINOKINASE™, a new pharmacological compound which includes a component improving fibrinolysis and a component controlling edema (Fujii et al., Nihon kessen shiketsu shi, 43:1124 (1994); Sumi et al., Acte Haematol, 84:139-11 (1990); Editorial, Journal Suisse de médecine globale, 1/95:69-73 (1995); Petrassi et al., Phytomedicine, 7(5):383-88 (2000), the disclosures of which are hereby incorporated by reference in their entireties).

In some embodiments of the invention, compositions useful for preventing edema, DVT, and PE are provided. In some embodiments of the invention, a combination of an antioxidant and a fibrinolytic agent is used. In preferred embodiments of the invention, the combination comprises an antioxidant in combination with the fibrinolytic enzyme nattokinase. In other preferred embodiments, the combination comprises the enzyme nattokinase and an extract of pine bark. In the most preferred embodiments, the composition comprises the combination of the enzyme nattokinase and the antioxidant pine bark extract PYCNOGENOL®. The combination of these ingredients is coined “PINOKINASE™.” While PINOKINASE™ and its uses are among the preferred embodiments of the present invention, numerous other useful embodiments exist. The surprisingly effective combination of any suitable antioxidant and any suitable fibrinolytic agent, as disclosed herein, thus include numerous and varied embodiments of the invention, and descriptions herein of preferred embodiments directed to any particular antioxidant and/or any particular fibrinolytic agent are merely exemplary.

Dangers of DVT During Flight or Other Situations of Extended Immobilization

The dangers posed by DVT after long flights or other situations of extended immobilization are not minimal (Collin J., Lancet., 358:838 (2001); Reynolds M., Lancet., 358:838-9 (2001); Anderson R., Lancet., 358:837 (2001); Burnand et al., Lancet., 358:837 (2001); Bendz et al., Lancet., 358:837-8 (2001); Teenan et al., Br J Clin Pract., 46:165-6 (1992); Hosoi et al., Eur J Vasc Endovasc Surg, 24:235-8 (2002); Belcaro et al., Circulation, 106:II:721 (2002), the disclosures of which are hereby incorporated by reference in their entireties). The problem is particularly noticeable in high-risk individuals—one study found that the incidence of DVT in high-risk subjects was greater than 4% per long-haul flight (Belcaro et al., Angiology, 53:17-21 (2001); Belcaro et al., Angiology, 52:369-74 (2001), the disclosures of which are hereby incorporated by reference in their entireties). It has been suggested that during prolonged flights (24 hours) some 10% of passengers may be affected by DVT (Mendis et al., Bull World Health Organ., 80:403-6 (2002); Cesarone et al., Ediz Minerva Medica (2001); Scurr et al., Lancet, 357:1485-89 (2001); Belcaro et al., Angiology, 53(6):635-45 (2002) the disclosures of which are hereby incorporated by reference in their entireties). However, most after-flight DVTs are neglected as they are often (89%) asymptomatic (Cesarone et al., Angiology, 53:1-6 (2002); Belcaro G., Circulation, Suppl II, 104:II-528 (2001); Belcaro et al., J Am Coll Cardiol, 39, Suppl 5:212A (2002), the disclosures of which are hereby incorporated by reference in their entireties). Patients with a history of thrombosis and chronic venous insufficiency are at particularly higher risk of developing new episodes (Lethagen, Lakartidningen 98:4063 (2001), the disclosure of which is hereby incorporated by reference in its entirety). Approximately 56% of patients with a documented DVT had previous possible episodes of thrombosis (Belcaro G., Circulation, Suppl II, 104:II-528 (2001); Belcaro et al., J Am Coll Cardiol, 39, Suppl 5:212A (2002).

Our recent prevention study indicates that there is a significant risk (5-7%) of thrombotic events in high-risk individuals during longer flights, and that most thrombotic events may be prevented by compression (Belcaro et al., Cl Appl Thr Heost, in press (2003), the disclosure of which is hereby incorporated by reference in its entirety). A conflicting report showed no observation of DVT but an increase in D-Dimer in a percentage of subjects (Jacobson et al., Sout Af Med Journal, in press (2003)). The presence of thrombotic disease in an individual may be associated with elevated levels of D-dimer, and assays are available to detect levels of D-dimer. However, in the Jacobson publication, risk categories were not separated and there was no prophylaxis. Venous disease, edema and DVT (Nicolaides A N, Circulation, 102:126-63 (2000); Belcaro et al., Venous Disorders: a manual of diagnosis and treatment, Saunders, Cambridge (1996), the disclosures of which are hereby incorporated by reference in their entireties) are very common observations and some 35% of subjects flying for more than 10 hours may have venous disease or some type of edema. (Belcaro et al., Cl Appl Thr Heost, in press (2003); Nicolaides A N, Circulation, 102:126-63 (2000); Belcaro et al., Venous Disorders: a manual of diagnosis and treatment, Saunders, Cambridge (1996), the disclosures of which are hereby incorporated by reference in their entireties). The classification of risk categories for venous thrombosis (Dalen et al., Sixth ACCP Consensus Conference on Antithrombotic Therapy, Chest, 119;1 (Suppl) (2001); “Prevention Of Venous Thromboembolism,” International Consensus Statement. Edition 2002, Med-Orion, London (2002), the disclosures of which are hereby incorporated by reference in their entireties), is well defined but it is possible that for conditions such as long-flights risk categories may be adjusted to different standards.

Of course, the swelling, DVT, and PE caused by long term immobility is not limited to airline flights. For example, the occurrence of a high incidence of DVT (with some episodes of PE) in pilgrims traveling by bus has recently been observed (Cesarone et al., European Venous Forum Web Journal, 1,1 (2003), the disclosure of which is hereby incorporated by reference in its entirety). Travel situations where cramped sitting positions may be necessary for several hours at a time include but are not limited to bus travel, car travel, boat travel, and the like. Other situations where immobilization for several hours may cause similar symptoms include but are not limited to test taking, studying, reading, office work, incarceration, and the like. Additionally, invalids, or persons otherwise temporarily or permanently immobilized in cramped positions for extended periods may also experience edema, swelling, DVT, and PE. The combination of any of these immobilization periods may further exacerbate the risk of swelling, DVT, and PE. For example, in some situations, a trip by airline may additionally include long periods of immobilization while traveling to get to the airport, followed by long periods of immobilization waiting for the flight. Complicated travel plans could result in the individual being forced to sit in an immobilized, cramped state for well over 24 hours.

Some relief can be obtained by adherence to preventive measures such as elastic stockings and anti-thrombotic prophylaxis with low molecular weight heparin (LMWH). (Belcaro et al., Angiology, 53:17-21 (2001); Belcaro et al., Angiology, 52:369-74 (2001); Cesarone et al., Angiology, 53:1-6 (2002); Belcaro G., Circulation, Suppl II, 104:II-528 (2001); Belcaro et al., J Am Coll Cardiol, 39, Suppl 5:212A (2002). An evaluation of DVT prevention with stockings (Belcaro et al., Angiology, 53:17-21 (2001)) has shown that stockings decrease DVT incidence during long-haul flights. The drug coumadin is also used to increase bleeding times to prevent blood clots. Other measures that may lessen the risk of DVT during long flights include periodic standing, stretching, exercising, drinking fluids, and avoiding tight clothing.

Fibrinolytic Agent

In preferred embodiments, the composition taken to prevent or control DVT, edema, and PE contains the fibrinolytic enzyme nattokinase. Nattokinase was originally extracted from a traditional Japanese fermented cheese-like food made from the combination of boiled soy beans and Bacillus subtilis, natto, which has been consumed orally for at least 400 years. B. subtilus has been given GRAS (Generally Regarded as Safe) status in the USA and has been marketed as a natural biological control product in many countries. Natto and nattokinase have been found to have fibrinolytic activity (Sumi H., 1:49, JTTAS, Tokyo (1994); Sumi et al., Nihon Shokukakoushi, 43:139-40 (1996); Sumi et al., Experientia, 43:1110-12 (1987); Sumi et al., Fibrinolysis, 6:86-9 (1992), the disclosures of which are hereby incorporated by reference in their entireties). Oral administration of nattokinase produces a significant enhancement of the fibrinolytic activity in the plasma, as indicated by measurement of fibrinolytic parameters and the endogenous product of tissue plasminogen activator.

Nattokinase is a serine proteinase having approximately 275 amino acids and a molecular mass of approximately 27.7 kDa in its mature form (Fujita et al., Biochem Biophys. Res. Commun. 197:1340-1347 (1993), the disclosure of which is hereby incorporated by reference in its entirety)). Nattokinase has some sequence similarity to members of the subtilisin family of proteins (Urano et al., J. Biol. Chem. 276:24690-24696 (2001), the disclosure of which is hereby incorporated by reference in its entirety).

The nattokinase enzyme used in preferred embodiments of the invention may be extracted from natto, or may be produced, for example, in commercial fermentors, or may be engineered to be expressed in sources other than the original organism. Methods for purifying nattokinase are described, for example, in Fujuta et al., (1993), supra. Methods for purifying nattokinase can also be found using general protein purification methods as described, for example, in Scopes, R., Protein Purification, Springer-Verlag, NY, (1992)). Alternatively, nattokinase can be obtained commercially through suppliers such as Aidan Products, LLC (Tempe, Ariz.), Nutricology, Inc. (Hayward, Calif.), or Nutriscience Innovations (Farfield, Conn.).

Nattokinase nucleic acid and protein sequences from Bacillus subtilis can be found in publicly available databases such as Genbank. An exemplary protein sequence of the mature nattokinase polypeptide is Genbank Accession No. CAC41625 (SEQ ID NO: 1), the disclosure of which is incorporated herein by reference in its entirety). The mature nattokinase enzyme is cleaved from the pro-protein sequence of approximately 381 amino acids. Exemplary amino acids of the Bacillus subtilis nattokinase pro-protein sequence include Genbank Accession Nos. AA065246 (SEQ ID NO: 2), JH0778 (SEQ ID NO: 3), and AAK54130 (SEQ ID NO: 4), the disclosures of which are incorporated herein by reference in their entireties). Exemplary Bacillus subtilis nucleic acid sequence encoding the pro-protein include, for example, Genbank Accession Nos. AY219901 (SEQ ID NO: 5), AJ314856 (SEQ ID NO: 6), and AF368283 (SEQ ID NO: 7), the disclosures of which are incorporated by reference herein in their entireties). In some embodiments, the nucleic acid sequence encoding the nattokinase enzyme or its pro-protein sequence may be engineered to be expressed from other bacterial species, or may be engineered to be expressed in yeast or plant species.

In some embodiments of the invention, other fibrinolytic agents may be used. Examples of such agents include but are not limited to urokinase, subtilisin, and plasmin. The fibrinolytic agents may be derived, for example, from plant, animal or microbial sources. Preferably, the fibrinolytic agent enzyme is derived from natural sources.

Antioxidant

In most preferred embodiments, the antioxidant used is PYCNOGENOL®. PYCNOGENOL® is a water extract from the bark of the French maritime pine (Pinus maritima). The extract contains a natural blend of constant proportions of bioflavonoids including catechin, epicatechin, taxifolin, oligomeric proanthocyanidnins and phenolic fruit acids (ferulic acid and caffeic acid). PYCNOGENOL® is a powerful anti-edema compound and increases capillary wall resistance, makes them less permeable, contributes to the control of edema and has anti-inflammatory effects (Gabor et al., Phlebologie, 22:178-182 (1993), the disclosure of which is hereby incorporated by reference in its entirety). PYCNOGENOL® is effective in treating edema in venous insufficiency (Schmidtke et al., Journal Suisse de médecine globale, 3/95:114-115 (1995)), and has also been shown to reverse symptoms of venous insufficiency (Editorial, Journal Suisse de médecine globale, 1/95:69-73 (1995); Petrassi et al., Phytomedicine, 7(5):383-88 (2000), the disclosures of which are hereby incorporated by reference in their entireties).

In some embodiments of the invention, the antioxidant is therefore an aqueous extract prepared from French maritime pine. In other embodiments, the antioxidant is pine bark extract prepared from any type of pine species.

In further embodiments, antioxidants from other sources may be used. Examples of antioxidants which may be useful for the invention include but are not limited to vitamin C, vitamin E, catechins, carotenoids, flavonoids, coenzyme Q10 (ubiquinone), isoflavones, phenylpropanoids, polyphenols, tocopherols, alpha tocopherol, selenium, magnesium, α-lipoic acid, tert-butylhydroquinone (TBHQ), butylated hydroxyanisole (BHA), butylated hydroxytoluene (BHT), tocotrienols, ascorbic acid, resveratrol, and the like.

In some embodiments of the invention, the antioxidant may be a mixture of plant-derived material, or may be an oil or aqueous extract of plant material. Examples of natural antioxidant mixtures or preparations derived from plants include but are not limited to extracts of pine bark, oleoresins or extracts of spices (such as rosemary extract), tea extracts, grape seed extracts, antioxidants from fruit skin and seeds, other plant-based extracts having antioxidant activity, and the like.

Pharmaceutical Compositions

Pharmaceutical compositions of the fibrinolytic agent plus antioxidant compounds of the invention for use in accordance with the present invention can be formulated in a conventional manner using one or more physiologically acceptable carriers or excipients. Thus, the compounds and their physiologically acceptable salts and solvates can be formulated for administration by inhalation or insufflation (either through the mouth or the nose) or oral, buccal, parenteral or rectal administration. Preferably, the formulation is in tablet form. The formulation may also take the form of, for example, a lozenge, a chewing gum, a liquid, a gel, a solid, a powder form, and the like.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate. talc or silica); disintegrants (e.g., potato starch or sodium starch glycolate); or wetting agents (e.g., sodium lauryl sulfate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g. methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound. For buccal administration the compositions can take the form of tablets or lozenges formulated in conventional manner.

Therapeutic formulations of the fibrinolytic agent plus antioxidant compounds of the invention may be prepared for storage by mixing the compounds having the desired degree of purity with optional physiologically acceptable carriers, excipients, or stabilizers (Remington: The Science and Practice of Pharmacy, 19th Edition, Alfonso, R., ed, Mack Publishing Co. (Easton, Pa.: 1995), the disclosure of which is hereby incorporated by reference in its entirety), in the form of lyophilized cake or aqueous solutions. Acceptable carriers, excipients or stabilizers are nontoxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

Preferably, the fibrinolytic agent plus antioxidant compounds of the invention will be formulated, dosed, and administered in a fashion consistent with good medical practice. Factors for consideration in this context include the level of edema, DVT or PE being treated, the clinical condition of the individual patient, the risk level of developing DVT or PE, the length of an airline flight or other form of long-term travel wherein the individual would be in need of treatment, the site of delivery of the compound, the particular type of compound, the method of administration, the scheduling of administration, and other factors known to medical practitioners. The “therapeutically effective amount” of such a compound to be administered will be governed by such considerations, and is the minimum amount necessary to prevent, ameliorate, or treat edema, DVT or PE. Such amount is preferably below the amount that is generally considered to be toxic to the host.

As a general proposition, the initial pharmaceutically effective amount of the fibrinolytic agent plus antioxidant compounds can be administered orally will be in an acceptable range of from about 0.05 or 0.1 to about 600, or 700, or 800, or 900, or 1,000 mg/kg of patient body weight per day, with the typical initial range of compound used being preferably from about 0.15 or 0.2 to about 20, 50, 100, 200, 300, 400, or 500 mg/kg/day, and more preferably from about 0.3, 0.5, 1, 2, 3, 5, or 6, to about 7, 8, 10, 12, 14, or 15 mg/kg/day.

When the antioxidant used is PYCNOGENOL®, an acceptable range of administration would be, for example, from about 2, 4, 8, 12, or 16, to about 1,800, 2,000, 2,500, 3,000, 4,000, or 5,000 mg per dosage, preferably from about 20, 25, or 30, to about 800, 1,000, 1,200, 1,400, or 1,600 mg per dosage, more preferably from about 50, 100, 150, 200, or 250 to about 300, 350, 400, 450, 500, or 600 mg per dosage.

The fibrinolytic agent may have an activity of, for example, a range of from about 50, 70, or 90 to about 20,000, 30,000, 40,000, or 50,000 fibrinolytic units per dosage. Preferably, the fibrinolytic agent has a range of from about 100, 200, 300, 400, or 500 to about 6,000, 8,000, or 10,000 fibrinolytic units per dosage. More preferably, the fibrinolytic agent has an activity of in the range of from about 750, 1,000, 1,500, 2,000, or 2,500 to about 3,000, 4,000, or 5,000 units per dosage.

Preferably, the fibrinolytic agent used is nattokinase having activity in the range of from about 50, 70, or 90 to about 20,000, 30,000, 40,000, or 50,000 fibrinolytic units per dosage. More preferably, the nattokinase has a range of from about 100, 200, 300, 400, or 500 to about 6,000, 8,000, or 10,000 fibrinolytic units per dosage. Most preferably, the fibrinolytic agent has an activity in the range of from about 750, 1,000, 1,500, 2,000, or 2,500 to about 3,000, 4,000, or 5,000 units per dosage.

When the composition is administered to prevent or reduce problems associated with long term flights, for example, lower extremity edema, DVT, or PE, the dosage can be delivered by a single dose given, for example, about 12, 10, or 8, to about 4, 2, or 0 hours before the flight. More preferably, the dosage is administered within 6 hours of the flight, and most preferably, within 4 hours of the flight. Alternatively, the dosage can be given during the flight. Embodiments of the invention include administering the composition both before and during the flight, before the flight only, or during the flight only. Additionally, the dosage can be administered several times throughout the flight, for example, every 4 hours, or every 3 hours, or every 2 hours, or every 1 hour, or every 30 minutes. Further, in some circumstances, use of the formulation may be advisable after the flight. For example, post flight administration of the formulation can assist in relieving the level of edema that has developed during the flight. It will be understood that the references to flight can be substituted with any other situation involving prolonged periods of inactivity, for example, confinement or enforced inactivity.

As noted above, however, these suggested amounts of compound are subject to a great deal of therapeutic discretion, including the individual type of compound being used. The key factor in selecting an appropriate dose and scheduling is the result obtained, as indicated above. For example, the compound may be optionally formulated with one or more agents currently used to prevent or treat DVT or PE. The effective amount of such other agents depends on the amount of the compound present in the formulation, the clinical level of the DVT or PE, and other factors discussed above. These are generally used in the same dosages and with administration routes as used hereinbefore or about from 1 to 99% of the heretofore employed dosages.

One of skill in the art would be able to select an appropriate dosage of the combination of an antioxidant and a fibrinolytic agent, for example, by preparing a formulation with varying amounts of the antioxidant and the fibrinolytic agent, then testing the dosage on individuals during a long-haul light. The effectiveness of the dosage can be determined by comparing the edema score, for example, as described in Example 8, prior to and after the flight. Formulations capable of decreasing the amount of ankle or leg swelling, as compared to that of control individuals taking placebo formulations, can then be selected for use. Preferred formulations are those which result in the least amount of leg swelling, ankle swelling, or other edema scoring criteria.

Use of the Combination of an Antioxidant (Such as PYCNOGENOL®) Plus a Fibrinolytic Agent (Such as Nattokinase) to Prevent Edema, Superficial and Deep Vein Thrombosis

To evaluate the effect of the combination of the antioxidant PYCNOGENOL® plus a fibrinolytic agent nattokinase on the development of edema, superficial and deep vein thrombosis (DVT) prophylaxis in high-risk individuals during long-haul flights (7-8 hours), a study was undertaken (Examples 2 through 11). Subjects were administered tablets containing both PYCNOGENOL® and nattokinase, as described in Examples 2 and 3. The study group was chosen as described in Example 4. The method used for ultrasound scanning to study the venous system is detailed in Example 6. The method for determining D-dimer and fibrinogen levels is detailed in Example 7. Edema evaluation methods are described in Example 8. Edema scoring criteria is shown below in Table 1.

TABLE 1
Parameters and items considered in the evaluation of edema.

Scale

| | 0 | 1 | 2 |
|---|---|---|---|
| 1 Edema test | 0-<1 | >1-2 | >2 |
| 2 Ankle circumference (cm)* | 0-1 | >1-2 | >2 |
| 3 Volume (mL)* | 0-2 | >2-5 | >5 |
| 4 Subjective swelling | 1-10 | 1-2 | >2 |
| 5 Discomfort | 1-10 | 1-2 | >2 |
| Max. score | 0 | 5 | 10 |

Worst case, 10; no edema, 0.
*Difference before - after.

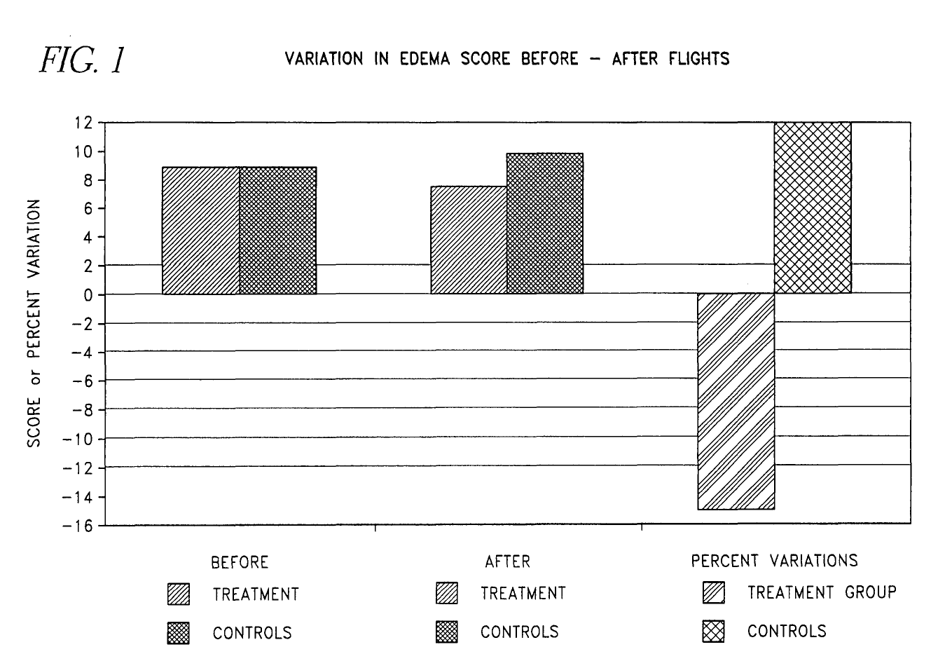

At inclusion edema was comparable in the two groups. After flights there was an increase in score in controls (+12%) in comparison with a decrease (−15%) in the PYCNOGENOL® plus nattokinase group, as shown in Table 2, below, and in FIG. 1 (the difference in variation was statistically significant). In conclusion, the tablets containing PYCNOGENOL® plus nattokinase were effective in reducing and controlling edema in high-risk subjects in long flights.

TABLE 2
Edema variation.

| Score | Treatment | Control | Difference |
|---|---|---|---|
| Before | 8.88 (SD 1.2) | 8.7 (SD 1) | ns |
| After | 7.54 (SD 0.8) | 9.8 (SD 0.5) | p < 0.05 |
| % Variation | −15 | +12 | p < 0.02 (27%) |

Analysis of Thrombotic Events After a Long-Haul Flight

The results of the study are summarized in Table 3, below. Interestingly, no DVT was observed in the treatment group. However, in the control group, 5 subjects (5.43%) had a DVT, and there were also 2 superficial thromboses (7 events in 92 subjects=7.6%). The 4 females who had a thrombotic event in the control group (3 DVT, 1 SVT) were under low-dose oral contraceptives for at least 8 months prior to the flight. No statistically significant difference was observed in the events distribution between men and women (3 to 4; 3 DVT, 1 SVT in women).

TABLE 3
Results table.

| | CON | Treat | Total | pValue |
|---|---|---|---|---|
| Selected subjects | 114 | 110 | 224 | |
| BIncluded | 103 | 101 | 204 | ns |
| Completing the Study | 92 | 94 | 186 | |
| Lost | 11 | 7 | 18 | |
| DVT | 5 | 0 | 5 | <0.025 |
| SVT | 2 | 0 | 2 | <0.05 |
| Events (%) | 7.6 | 0 | 7.6 | <0.025 |
| ITT* (failures) | 18/92 | 7/94 | 25/186 | <0.05 |
| % | 19.6 | 7.4 | 13.44 | <0.05 |

Of the 300 pre-selected subjects, 224 were actually included (76 were excluded for several reasons); 186 completed the study (18 were lost at the end of the flight for non-medical problems, mainly connections).
*ITT: Intention to treat analysis detects 18 failures in the control group (11 lost to follow up + 7 thrombotic events) out of 92 subjects (19.6%) versus 7 failures (7.4%, all lost, no events) in the treatment group (p < 0.05).

The presence of DVT was associated with edema at the end of the flight. It is possible that the development of edema was relevant factor in producing DVT causing vein compression. In conclusion, the combination of pine bark extract and nattokinase is effective in reducing the incidence of flight-related DVT and in controlling edema in high-risk subjects during long-haul flights.

Interestingly, it is quite possible that most of the signs and symptoms of swelling observed in our study would have been completely neglected by the study subjects who considered some form of swelling almost normal after sitting for so long. However, considering the health risks such extended swelling may pose, especially in high risk individuals, efforts to control edema may play an important role in decreasing the incidence of thrombotic events.

Regarding Individuals at High Risk for DVT

The incidence of DVT in high-risk individuals may be high (4-10%), according to risk level, flight length, or other conditions. Prophylaxis is advisable particularly for high-risk subjects. (Belcaro G., Circulation, Suppl II, 104:II-528 (2001); Belcaro et al., J Am Coll Cardiol, 39, Suppl 5:212A (2002); Anonymous, American Heart Association Scientific Sessions, 3:8 (2001), the disclosures of which are hereby incorporated by reference in their entireties). Elastic stockings are an effective solution for prophylaxis. In higher-risk subjects enoxaparin is effective in decreasing risk of DVT, at a relatively higher cost and with a limited risk of side effects. Exercise during flights—if and when possible—diet suggestions, less baggage on board (to keep free leg space) and larger empty spaces on planes may help. Suggestions from physicians not to travel or to travel in a different way are very important in conditions of particularly high risk. All patients with a recent history of thrombosis, or with chronic venous insufficiency are at higher risk of DVT (Cesarone et al., Ediz Minerva Medica (2001); Scurr et al., Lancet, 357:1485-89 (2001), the disclosures of which are hereby incorporated by reference in their entireties). The average population flying on planes is different from our selected samples (i.e. we have excluded subjects with cardiovascular disease requiring drug treatment, those particularly handicapped or very old) and may be prone to more thrombotic events. Therefore these results, when extrapolated to the general flying population may find a higher incidence of thrombotic events and a better cost-efficacy ratio of prophylaxis. The pine bark extract/nattokinase combination present in FLITE TABS™ may offer a very important option for prophylaxis without increasing risks.

Other Treatments That May be Useful in Combination With the Antioxidant/Fibrinolytic Agent Composition of the Invention

The use of the combination of antioxidant and fibrinolytic agent of the invention may, in some situations, be combined with other treatments. For example, the PINOKINASE™ treatment may be combined with the use of elastic stockings, exercise, diet changes, and other treatments. This may be particularly useful in high risk patients.

In conclusion, the constituents of PINOKINASE™ have reduced symptoms associated with traveling on long-haul flights greater than either ingredient alone. Those symptoms included lower extremity edema, aching, and discomfort. As shown herein, the combination of the two ingredients can reduce edema and edema-related symptoms, and prevent DVT in high-risk individuals. These results may be even more important and significant when extrapolated to the general flying population. Thus, in preferred embodiments, the combination of PYCNOGENOL® and nattokinase offers an economic yet effective edema and DVT preventive option, without increasing health risks (i.e. due to the use of drugs) in the individual. In additional embodiments, the combination of an antioxidant and a fibrinolytic agent can also be effective to prevent or reduce edema, DVT, and PE.

The above disclosure generally describes the present invention. A more complete understanding can be obtained by reference to the following specific examples which are provided herein for purposes of illustration only and are not intended to limit the scope of the invention.

### EXAMPLE 1

### General Tablet Formulation for the Combination of an Antioxidant and a Fibrinolytic Agent

Tablets and capsules are prepared using the following ingredients. PYCNOGENOL® at 20 to 2000 mg, and nattokinase having activity in the range of 100 to 10,000 fibrinolytic units (as such units are measured using standard methodology that is known to the art). The tablets and capsules are prepared by conventional means with pharmaceutically acceptable excipients including binding agents. Additionally, some of the tablets are coated with a gelatin coating prepared by methods well known in the art.

Other tablets and capsules are prepared using grape seed extract, rosemary extract, and vitamin C, in various combinations with fibrinolytic agents urokinase, subtilisin, and plasmin, thus producing a number of different formulations. The dosage of each combination is selected based upon the known safe maximum dosage of the selected antioxidant and the selected fibrinolytic agent, and the dosage employed is between the safe maximum and the typical recommended dosage.

### EXAMPLE 2

### Formulation of PINOKINASE™ Tablets Used in Long-Haul Flight Trial

The formulation used for the FLITE TABS™ used in the long-haul flight trial described below in Examples 3 through 11 is as follows: Each capsule contains approximately 150 mg PINOKINASE™, which is a blend of 50 mg of nattokinase (at a concentration of approximately 20,000 fibrinolytic units per gram), combined with approximately 100 mg of PYCNOGENOL®, a trade name for a specific type of pine bark extract (Fujii et al., Nihon kessen shiketsu shi, 43:1124 (1994); Sumi et al., Acte Haematol, 84:139-11 (1990); Editorial, Journal Suisse de médecine globale, 1/95:69-73 (1995); Petrassi et al., Phytomedicine, 7(5):383-88 (2000), the disclosures of which are hereby incorporated by reference in their entireties). The capsules additionally contained rice flour and were coated with a gelatin capsule.

### EXAMPLE 3

### Administration of FLITE TABS™ Containing PINOKINASE™

Subjects consumed two capsules of the composition described in Example 2 approximately 2 hours before flights, along with 250 ml of water. Subjects also took two capsules approximately six hours later, along with 250 ml of water. Placebo capsules were administered accordingly to the control group with the same amount of fluid.

### EXAMPLE 4

### Determnination of Study Subjects

300 subjects at high-risk for DVT were contacted and pre-included after informed consent; 76 subjects were excluded on the basis of several considerations—use of anticoagulant or anti-thrombotic drugs (22 subjects), cardiovascular treatments (Belcaro et al., Angiology, 53(6):635-45 (2002)), difficulty in wearing stockings (AMA Commission on Emergency Services, “Medical aspects of transportation aboard commercial aircraft,” JAMA 247:1007-1011 (1982)), and possible low compliance (Sarvesvaran R., Med Sci Law, 26:35-38 (1986)). We included 204 subjects (103 controls group and 101 in the treatment group). The subjects were randomized into two groups to evaluate prophylaxis with specific stockings in 7-8-hour flights (New York-London or London-New-York). High-risk criteria for DVT are those indicated in previous studies (Belcaro et al., Angiology, 53:17-21 (2001); Belcaro et al., Angiology, 52:369-74 (2001); Cesarone et al., Angiology, 53:1-6 (2002); Belcaro G., Circulation, Suppl II, 104:II-528 (2001); Belcaro et al., J Am Coll Cardiol, 39, Suppl 5:212A (2002); Anonymous, American Heart Association Scientific Sessions, 3:8 (2001), the disclosures of which are hereby incorporated by reference in their entireties), such as previous episodes of DVT or superficial vein thrombosis, coagulation disorders, severe obesity or limitation of mobility due to bone or joint problems, neoplastic disease within the previous two years, clinical cardiovascular disease, large varicose veins. Further, subjects taller than 190 cm and heavier than 90 kg were excluded.

Exclusion criteria were clinical diseases requiring medical treatment, severe bone/joint problems or limited mobility, uncontrolled diabetes, severe hypertension, obesity, recent thrombosis (less than 6 months), presence of thrombi, and increased D-Dimer level at the pre-flight examination.

Flight duration was on average 7 hours and 44 minutes (SD 34 min). Out of the 103 included subjects in the control group and 101 in the treatment group (204), 92 controls and 94 treated subjects (186) completed the study. Drop-outs were due to low compliance to the protocol or flight-connection problems. Age/sex distributions were comparable in the two groups.

### EXAMPLE 5

### Side Effects and Tolerability of Administered Capsules

Administration of PYCNOGENOL® has been shown to have very few side effects. Previous clinical studies on more than 2,000 patients have shown that very rarely, mild side-effects such as gastro-intestinal upsets may occur upon oral administration of PYCNOGENOL®. In this study, the tolerability of FLITE TABS™ was very good. There were no major complaints or side effects, and no subject stopped the prophylaxis plan. The compliance to treatment was very good (98% of the capsules were correctly used).

### EXAMPLE 6

### Ultrasound Scanning Protocol (Before/After Flights)

Ultrasound scanning was used to study the venous system by compression of the major veins (femorals, popliteal and tibials and the superficial veins) (Belcaro et al., Imperial College Press, London (1999); Belcaro et al., Imperial College Press, London (2001), the disclosures of which are hereby incorporated by reference in their entireties). The scanning was performed within 90 minutes before the flight and just after the flights (within 90 minutes), using Sonosite scanners with a 7.5-13 MHz, high-resolution, linear probe (Sonosite, Bothell, Wash., USA).

### EXAMPLE 7

### D-Dimer and Fibrinogen Tests

In general, thrombotic disease may be associated with elevated levels of D-dimer. Therefore, D-dimer and fibrinogen tests were performed before (within 12 hours) flights and within 2 hours after the flight (Dade Dimertest, Latex Test, Boehring, Germany). The D-dimer level of subjects in the study fell within normal values (<200 ng/mL) before inclusion. The test at arrival was not possible in 6 subjects (due to connections problems). All tests were performed within 2 hours after the flight (average 69 min; SD 23 min). D-dimer test results were within the normal range after the flight and no significant difference between mean values measured in subjects with ultrasound-detected DVT and those without DVT was observed.

The D-dimer assay can be performed in a few minutes and it has no cross-reactivity with fibrinogen or its breakdown products. When interpreting the results of D-dimer tests, several important considerations are typically taken into account. The precise level of cross-linked D-dimer circulating in the blood at any given time will depend on a number of parameters, including time elapsed since a thrombotic event, initial clot size, rate of fibrinolysis, the presence of alternative fibrin sites, assay differences, and differences in the specificity of the monoclonal antibody used in the assay.

Fibrinogen values were also measured, and found to be within the normal range before and after the flights and there were no significant differences between non-thrombotic and thrombotic subjects after the flight.

### EXAMPLE 8

### Evaluation of Edema

The edema scoring criteria are shown in Table 1. This combined edema score was developed to assess in a quantifiable and reproducible way edema and swelling. (Cesarone et al., J Cardiovasc Pharmac Therapeutics, 7 (Suppl I) S17-20 (2002); Thulesius O., Lakartidningen, 80(17):1791-801 (1983); Cesarone et al., Panmin Med, 41:10-14 (1999), the disclosures of which are hereby incorporated by reference in their entireties). The score is based on combined evaluation of parametric data (edema tester, variations in ankle circumference in cm, volume measurements in ml or in percent variation of the baseline volume) combined with the subjective assessments of swelling and discomfort measured on an analogue scale line. Items 4 and 5 are based on a scale line (range 0 to 10) defined by the study subjects before and after the flights. The edema tester (ACI-Medical, Ca, USA) is a device developed to assess edema in a semi-quantitative way. The device is applied at the internal perimalleolar region underneath a sphygmomanometer, with its distal edge 2-3 cm proximal to the medial malleolus; pressure is applied for 3 minutes (constant pressure of 50 mmHg). Pressure on the tester produce skin marks which are related to the presence and quantity of edema at the perimalleolar region. The edema tester had been studied and validated and previous studies indicate good reproducibility in standardized conditions (Cesarone et al., Panmin Med, 41:10-14 (1999), the disclosure of which is hereby incorporated by reference in its entirety). Ankle circumference was measured with a tape at the smallest ankle diameter. This method can measure with accuracy differences in variations of size >1 cm. Volume variations are measured with water displacement (a plexiglas leg-shaped chamber with a parallel 2 mm diameter tube connected with the main water chamber). This method (Thulesius O., Lakartidningen, 80(17):1791-801 (1983), the disclosure of which is hereby incorporated by reference in its entirety) can accurately measure water displacement (and its variations due to increased leg volume) with a range of accuracy of <2 ml (Cesarone et al., J Cardiovasc Pharmac Therapeutics, 7 (Suppl I) S17-20 (2002); Belcaro et al., Imperial College Press, London (2001), the disclosures of which are hereby incorporated by reference in their entireties). The maximum calf size is carefully measured before volume measurements and marked onto the skin. The leg is immersed in water and the water level—and its displacement—are measured at the maximum calf circumference. The leg volume before the flight is arbitrarily considered to be 100% and any increase in volume is therefore measured as a percentage.

### EXAMPLE 9

### Effect of PYCNOGENOL®/Nattokinase Combination on Edema After a Long-Haul Flight

The change in edema score, as scored using the methods described in Example 8 and in Table 1, is shown in Table 2. Edema prior to flight was comparable in the two groups (Table 2, FIG. 1). After flights, there was an increased score in controls (+12%) in comparison with a decrease (−15%) in the treatment group. The difference in variation was significant (P<0.02). In the control group 89% of subjects had a clear increase in ankle circumference and volume which was clinically evident and associated to some degree of discomfort. The control of edema with FLITE TABS™ was significant considering parametric data (circumference, volume) and non-parametric (analogue scale line) observations. In included subjects the average maximum calf size was 39.4 cm(SD 2) cm in men and 33 (SD 1.1) in women. The height of the maximum diameter was 38(SD 1.1) cm in men and 34(SD 1) in women. The average volume before flights was 2212 (SD 19) ml in men, 1998 (SD 12) in women. The minimum ankle circumference was 22.2 (SD 2) cm in men and 18.6 (SD 2) in women. Subjects selected for the study were average for weight and height. The results, shown in Table 2 and shown graphically in FIG. 1, show that the PINOKINASE™ treatment was effective in lowering the post-flight edema score.

### EXAMPLE 10

### Analysis of Thrombotic Events After a Long-Haul Flight

Statistical analysis was performed using non-parametric tests (Mann-Whitney U-test) and the analysis of variance considering event-free subjects completing the protocol. The specific incidence of thrombotic events (DVT or superficial thrombosis) was calculated and compared considering individuals and by intention-to-treat analysis.

In the control group of 92 subjects (mean age 49.8; SD 13; range 29-68; M:F=46:46), 5 subjects (5 limbs) had a DVT and 2 (2 limbs) had a superficial thrombosis (Table 1). The incidence of thrombotic events was 7.6%. In the treatment group of 94 subjects, (mean age 48; SD 12; range 27-69; M:F=48:45), no thrombotic event was observed. The difference in events incidence between the groups is significant (P<0.025). Intention to treat analysis detected 18 failures in the control group (11 lost to follow up+7 thrombotic events) out of 92 subjects (19.6%) in comparison with 7 failures out of 94 subjects (7.4%) in the treatment group (P<0.05).

### EXAMPLE 11

### Use of the Combination of PYCNOGENOL® and Nattokinase for the Prevention of Lower Extremity Edema on Long Term Airline Flights

An individual at risk for developing lower extremity edema prepares for a 10 hour airline flight. Two hours prior to the flight, the individual is given a tablet comprising a dose of 1,000 fibrinolytic units of nattokinase and 1,000 mg of PYCNOGENOL®. After 4 hours of flight time, and again at 8 hours into the flight, the individual takes an additional tablet containing the same dosage. After the flight, the individual does not experience symptoms of lower extremity edema, and the risk of airline flight induced formation of blood clots, DVT, or PE is lessened.

### EXAMPLE 12

### Administration of the Combination of an Antioxidant and a Fibrinolytic Agent Before, During, or After Periods of Extended Immobilization to Prevent or Lessen the Severity of Lower Extremity Edema, DVT, or PE

An individual at risk for developing lower extremity edema, DVT, or PE prepares for a 10 hour period of sitting in an confined, immobilized state. Two hours prior to the onset of the immobilization period, the individual is given a tablet comprising a dose of 1,000 fibrinolytic units of a fibrinolytic agent, and 10 mg to 5 g of an antioxidant composition. After several hours, the individual repeats the dosage. Optionally, the dosage can also be administered at or near the end of the immobilization period. The individual is tested prior to, during, and after the immobilization period. The combination of the antioxidant and the fibrinolytic agent is able to lessen edema measurements, as well as lessening the incidence of DVT and PE.

### EXAMPLE 13

### Use of the Combination of the Fibrinolytic Agent Urokinase and the Anti-Oxidant Grape Seed Extract for the Prevention of Lower Extremity Edema on Long Term Airline Flights

An individual at risk for developing lower extremity edema prepares for a 12 hour airline flight. Two hours prior to the flight, the individual is given a tablet comprising a dose of 1,000 fibrinolytic units of urokinase and 1.0 g of grape seed extract. After 4 hours of flight time, and again at 8 hours into the flight, the individual takes an additional tablet containing the same dosage. After the flight, the individual does not experience symptoms of lower extremity edema, and the risk of airline flight induced formation of blood clots, DVT, or PE is lessened.

### EXAMPLE 14

### Use of the Combination of the Fibrinolytic Agent Plasmin and the Anti-Oxidant Ascorbic Acid for the Prevention of Lower Extremity Edema During a 16 Hour Period of Traveling by Car

An individual at risk for developing lower extremity edema prepares for a 16 hour car trip. Upon leaving for the trip, the individual consumes a tablet comprising a dose of 800 fibrinolytic units of plasmin and 1.5 g of ascorbic acid. After 6 hours of travel, and again at 12 hours of travel, the tablet dosage is repeated. Upon reaching the destination, the individual does not experience symptoms of lower extremity edema, and the risk of formation of blood clots, DVT, or PE is lessened.

Although this invention has been described in terms of certain preferred embodiments, other embodiments which will be apparent to those of ordinary skill in the art in view of the disclosure herein are also within the scope of this invention. Accordingly, the scope of the invention is intended to be defined only by reference to the appended claims and any equivalents thereof. Each document cited herein is incorporated by reference in its entirety.

What is claimed is:

1. A pharmaceutical preparation comprising effective amounts of a fibrinolytic agent and an antioxidant.

2. The composition of claim 1, wherein the fibrinolytic agent is an enzyme selected from the group consisting of nattokinase, urokinase, subtilisin, and plasmin.

3. The composition of claim 1, wherein the antioxidant is selected from the group consisting of: vitamin C, vitamin E, a catechin, a carotenoid, a flavonoid, coenzyme Q10 (ubiquinone), an isoflavone, a phenylpropanoid, a polyphenol, a tocopherol, alpha tocopherol, selenium, magnesium, α-lipoic acid, TBHQ, BHA, BHT, a tocotrienol, ascorbic acid, resveratrol, a pine bark extract, an oleoresin, rosemary extract, tea extract, grape seed extract, and an antioxidant extract from fruit skin or from a seed.

4. A pharmaceutical preparation comprising effective amounts of pine bark extract and nattokinase.

5. The pharmaceutical preparation of claim 4, wherein said pine bark extract is prepared from French Maritime Pine, Pinus maritima.

6. A method for decreasing swelling of the lower extremities, edema, pulmonary embolism, or thrombosis, comprising:
administering a pharmaceutical preparation comprising a fibrinolytic agent and an antioxidant.

7. The method of claim 6, wherein said swelling of the lower extremities, edema, pulmonary embolism, or thrombosis results from confinement or enforced inactivity.

8. The method of claim 6, wherein said fibrinolytic agent is an enzyme.

9. The method of claim 8, wherein said enzyme is selected from the group consisting of urokinase, subtilisin, and plasmin.

10. The method of claim 8, wherein said enzyme is nattokinase.

11. The method of claim 6, wherein said antioxidant is selected from the group consisting of: vitamin C, vitamin E, a catechin, a carotenoid, a flavonoid, coenzyme Q10 (ubiquinone), an isoflavone, a phenylpropanoid, a polyphenol, a tocopherol, alpha tocopherol, selenium, magnesium, α-lipoic acid, TBHQ, BHA, BHT, a tocotrienol, ascorbic acid, resveratrol, an oleoresin, rosemary extract, tea extract, grape seed extract, and an antioxidant extract from fruit skin or from a seed.

12. The method of claim 6, wherein said antioxidant is pine bark extract.

13. The method of claim 12, wherein said pine bark is from French maritime pine (Pinus maritima).

14. The method of claim 12, wherein said extract is an aqueous extract.

15. The method of claim 12, wherein said extract comprises at least one component selected from the group consisting of: bioflavonoid, catechin, epicatechin, taxifolin, oligomeric proanthocyanidnin, phenolic fruit acid, ferulic acid, and caffeic acid.

16. The method of claim 7, wherein said confinement or enforced inactivity is due to one or more selected from the group consisting of: airline flight, bus travel, car travel, or train travel.

17. The method of claim 16, wherein said confinement or enforced inactivity is due to an airline flight.

18. The method of claim 17, wherein said airline flight is of a duration of greater than 6 hours.
